(19) United States
(12) Patent Application Publication
(10) Pub. No.: US 2016/0007923 A1
(43) Pub. Date: Jan. 14, 2016

(54) BIOLOGICAL SOUND COLLECTING DEVICE AND BIOLOGICAL SOUND COLLECTING METHOD

(71) Applicant: SHARP KABUSHIKI KAISHA, Osaka-shi, Osaka (JP)

(72) Inventor: Tomoaki YAMAMOTO, Osaka-shi (JP)

(21) Appl. No.: 14/770,858

(22) PCT Filed: Jul. 22, 2014
(86) PCT No.: PCT/JP2014/069324

§ 371 Date: Aug. 27, 2015

(30) Foreign Application Priority Data
Jul. 23, 2013 (JP) 2013-153005

(51) Int. Cl. A61B 5/00; A61B 7/02

(52) U.S. Cl. CPC A61B 5/6843; A61B 7/02; A61B 5/7445; A61B 2019/465

(57) ABSTRACT

A biological sound collecting device includes: a sound collecting unit configured to collect biological sound of a subject; a contact state detecting unit configured to detect a contact state of the sound collecting unit against a biological surface of the subject; a control unit configured to determine, based on the contact state detected by the contact state detecting unit, whether or not to have the sound collecting unit collect the biological sound; and an information notification unit configured to, in a case that the control unit determines not to have the sound collecting unit collect the biological sound, notify that the contact state detecting unit is not in contact with the biological surface of the subject.

### TECHNICAL FIELD

The present invention relates to a biological sound collecting device and a biological sound collecting method.

Priority is claimed on Japanese Patent Application No. 2013-153005, filed Jul. 23, 2013, the content of which is incorporated herein by reference.

### BACKGROUND ART

Stethoscopes are used as tools for auscultation in the medical field. For example, technique described in Patent Document 1 relates to a biological sound collecting device which performs processing on acoustic signals of a biological sound sensor attached to a body surface. The biological sound collecting device includes a noise extraction unit, a noise classification determination unit, and a noise countermeasure guide unit. The noise extraction unit extracts from an acoustic signal, a noise component contained in the acoustic signal. The noise classification determination unit classifies the extracted noise component into a plurality of noise types corresponding respectively to different mounted states of the biological sound sensor. The noise countermeasure guide unit presents to a user, information that urges an improvement in the mounted states associated with a result of the classification.

### CITATION LIST

### Patent Document

[Patent Document 1] Japanese Patent Application Laid-Open Publication No. 2012-200383

### DISCLOSURE OF THE INVENTION

### Problems to be Solved by the Invention

However, a user of a biological sound collecting device, such as a doctor, cannot accurately perform auscultation when the biological sound collecting device is tilted and pressed against a biological surface of a subject. For this reason, it is necessary to modify the pressing manner. However, even with the technology described in Patent Document 1, a user, such as a physician, cannot recognize the pressing state, thereby causing a possibility that the pressing manner of the biological sound collecting device cannot be modified. Thus, there is a possibility that a user of a biological sound collecting device cannot recognize a pressing state of the biological sound collecting device, and therefore cannot accurately auscultate biological sound of the subject.

One aspect of the present invention provides a biological sound collecting device and a biological sound collecting method capable of accurately auscultating biological sound of a subject.

### Means for Solving the Problems

One aspect of the present invention is a biological sound collecting device including: a sound collecting unit configured to collect biological sound of a subject; a contact state detecting unit configured to detect a contact state of the sound collecting unit against a biological surface of the subject; a control unit configured to determine, based on the contact state detected by the contact state detecting unit, whether or not to have the sound collecting unit collect the biological sound; and an information notification unit configured to, in a case that the control unit determines not to have the sound collecting unit collect the biological sound, notify that the contact state detecting unit is not in contact with the biological surface of the subject.

### Effects of the Invention

According to the biological sound collecting device and the biological sound collecting method according to one aspect of the present invention, it is possible to accurately auscultate biological sound of a subject.

### BRIEF DESCRIPTION OF THE DRAWINGS

FIG. 1 is a schematic view showing an example of usage of a biological sound collecting device according to a first embodiment of the present invention.

FIG. 2 is a schematic view showing an example of a pressing state of the biological sound collecting device against a biological surface according to the first embodiment of the present invention.

FIG. 3 is a schematic block diagram showing an example of a configuration of the biological sound collecting device according to the first embodiment of the present invention.

FIG. 4 is a schematic view showing an example of a notification aspect of state information in a presentation unit of the biological sound collecting device according to the first embodiment of the present invention.

FIG. 5 is a table showing an example of a relationship between a measured value measured by a contact state detecting unit and an estimated value calculated by a control unit according to the first embodiment of the present invention.

FIG. 6 is a flowchart showing an example of a pressing state presentation processing of the biological sound collecting device according to the first embodiment of the present invention.

FIG. 7 is a schematic block diagram showing an example of a configuration of a biological sound collecting device according to a second embodiment of the present invention.

FIG. 8 is a schematic block diagram showing an example of a configuration of a biological sound collecting device according to a third embodiment of the present invention.

FIG. 9 is an example of a top view when the biological sound collecting device according to the third embodiment of the present invention is viewed from an information notification surface, and is an example of a side view of the biological sound collecting device.

### MODE FOR CARRYING OUT THE INVENTION

### First Embodiment

Hereinafter, a first embodiment of the present invention will be described with reference to the accompanying drawings.

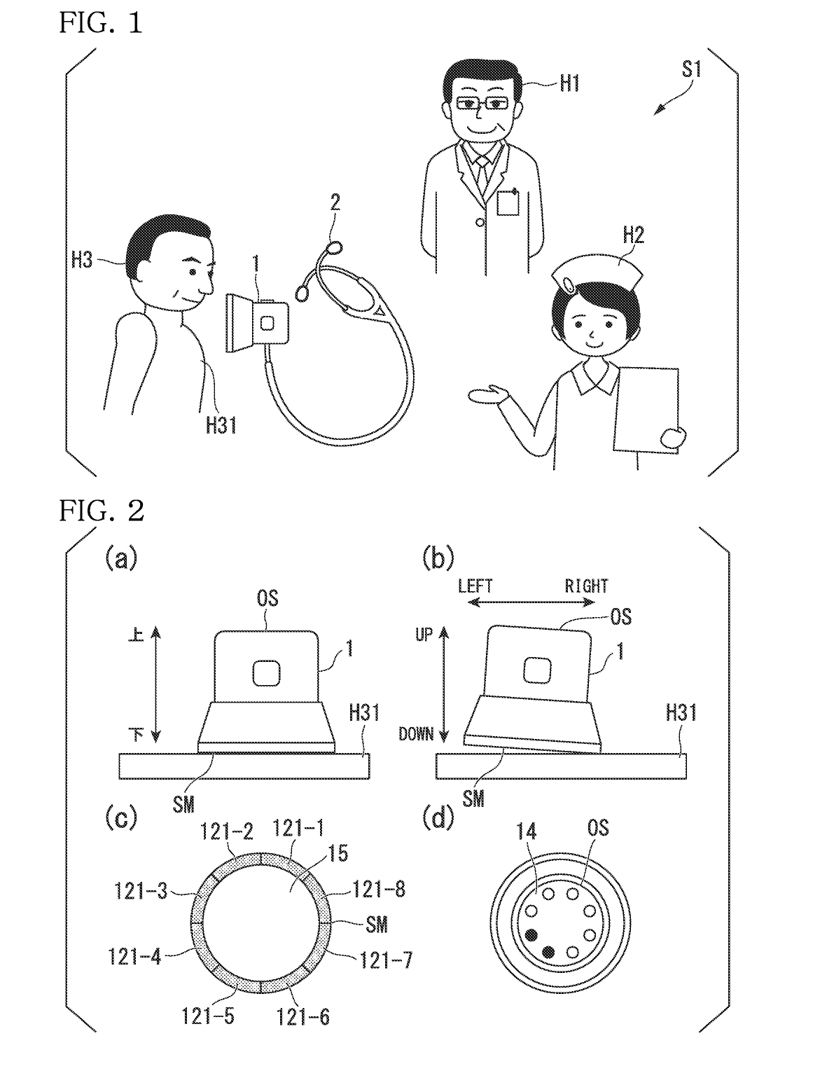

FIG. 1 is a schematic view showing an example of usage of a biological sound collecting device 1 according to a first embodiment of the present invention.

In a biological sound collecting situation Si in a biological sound collecting system, in order to collect biological sound (for example, heart sound) of a subject H3, a user has the biological sound collecting device 1 contact a part, to be inspected (for example, the left chest), of the subject H3. Here, the user includes, for example, a physician H1, a medical personnel H2, or the subject H3, a caregiver of the subject H3, and the like. When the user has the biological sound collecting device 1 contact a biological surface H31 of the subject H3 (for example, a surface of the left chest) to perform auscultation, the biological sound collecting device 1 detects a pressing state of the biological surface H31. If the pressing state is not proper, the biological sound collecting device 1 visually, aurally, and tactually notifies the user of the pressing state. Here, the proper pressing state is a state in which the contact surface is uniformly in contact with the biological surface H31. Detailed description will be described later with reference to FIG. 2.

If it is determined that the pressing state is proper, the biological sound collecting device 1 has a sound collecting unit 15 (not shown) collect biological sound of the subject H3 and output the collected biological sound of the subject H3 to, for example, an earphone 2 to be connected to the biological sound collecting device 1. The doctor H1 hears, using the earphone 2, the biological sound of the subject H3 collected by the biological sound collecting device 1, and examines the subject H3.

Here, for example, the user may collect biological sound of the subject H3 using only the biological sound collecting device 1, and output the collected biological sound of the subject H3 to an external device, such a computer, directly or via a network wiredly or wirelessly, for example. In this case, when the external device obtains the biological sound of the subject H3 from the biological sound collecting device 1, the doctor H1 may auscultate the biological sound of the subject H3 using the external device.

Additionally, the biological sound collecting device 1 and the earphone 2 may be connected wiredly or wirelessly.

FIG. 2 is a schematic view showing an example of a pressing state of the biological sound collecting device 1 against the biological surface H31 according to the first embodiment of the present invention.

The biological sound collecting device 1 has a contact surface SM at one end, and an information notification surface OS at the other end (see FIG. 2(a)). For example, a sound collecting unit 15 and a contact state detecting unit 12 are arranged on the contact surface SM. The contact state detecting unit 12 includes a plurality of sensors, for example, eight sensors, which are arranged on a periphery of the sound collecting unit 15. Additionally, an information notification unit 14 is disposed on the information notification surface OS.

FIG. 2(a) shows an example of a case where the user presses the biological sound collecting device 1 against the biological surface H31 and has the entire contact surface SM properly contact the biological surface H31. In this case, the information notification unit 14 of the biological sound collecting device 1 notifies the user that the biological sound collecting device 1 is properly pressed against the biological surface H31.

FIG. 2(b) shows an example of a case where the user cannot properly press the biological sound collecting device 1 against the biological surface H31. FIG. 2(b) shows a state in which a portion, for example, a lower right portion, of the contact surface SM of the biological sound collecting device 1 is pressed against the biological surface H31, but a lower left portion is not pressed.

FIG. 2(c) shows an example when viewed from the lower portion, that is, the contact surface SM side, of the biological sound collecting device 1.

On the contact surface SM, the sound collecting unit 15 is disposed at the center, and the sensors are arranged on the periphery of the sound collecting unit 15. For example, the periphery of the sound collecting unit 15 is divided into eight regions, and for example, a first sensor 121-1, a second sensor 121-2, a third sensor 121-3, . . . , and an N-th sensor 121-N (N is an integer) are arranged on the respective divided regions. Here, N=8 in the case of FIG. 2(c). In a state where the manner of the user pressing the biological sound collecting device 1 against the biological surface H31 is not proper, for example, in a state where the fourth sensor 121-4 and the fifth sensor 121-5 are not pressed against the biological surface H31, the information notification unit 14 notifies the user that the fourth sensor 121-4 and the fifth sensor 121-5 are not pressed against the biological surface H31.

FIG. 2(d) is an example when viewed from the upper portion, that is, the information notification surface OS side, of the biological sound collecting device 1.

The information notification unit 14 is disposed on the information notification surface OS. For example, on the information notification surface OS, a plurality of light emitting elements (for example, eight light emitting elements) are arranged as the information notification unit 14.

When the sixth sensor 121-6 and the seventh sensor 121-7 shown in FIG. 2(c) are not pressed against the biological surface H31, the information notification unit 14 turns on the light emitting elements corresponding to the sixth sensor 121-6 and the seventh sensor 121-7.

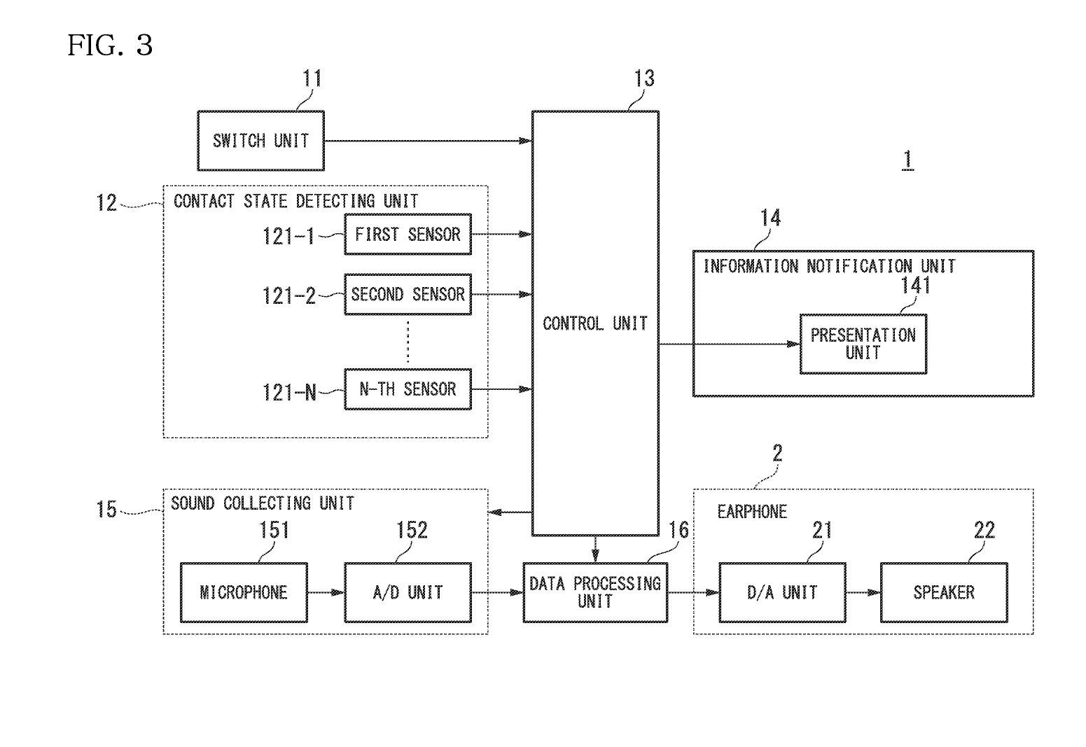

FIG. 3 is a schematic block diagram showing an example of a configuration of the biological sound collecting device 1 according to the first embodiment of the present invention.

The biological sound collecting device 1 includes a switch unit 11, a contact state detecting unit 12, a control unit 13, an information notification unit 14, a sound collecting unit 15, and a data processing unit 16. The contact state detecting unit 12 includes a first sensor 121-1, a second sensor 122-2, . . . , and an N-th sensor 122-N. The information notification unit 14 includes a presentation unit 141.

The sound collecting unit 15 includes a microphone 151 and an analog/digital conversion unit (A/D unit 152). An earphone 2 to be connected to the data processing unit 16 includes a digital/analog conversion unit (D/A unit 21) and a speaker 22.

The switch unit 11 is a main power supply, and outputs to the control unit 13, an operation signal input by user operation.

The contact state detecting unit 12 is, for example, a sensor of electrostatic capacity type. The contact state detecting unit 12 includes a plurality of sensors, such as a first sensor 121-1, a second sensor 121-2, . . . , and an N-th sensor 121-N (N is an integer). Each of the first sensor 121-1, the second sensor 121-2, . . . , and the N-th sensor 121-N has a similar configuration, and therefore will be described as the contact state detecting unit 12. The contact state detecting unit 12 measures, as a measured value, a change in capacitance caused by contacting the biological surface H31, and outputs the measured value to the control unit 13.

The control unit 13 determines based on the measured value received from the contact detecting unit 12 whether or not the biological sound collecting device 1 is pressed against the biological surface H31. Specifically, the control unit 13 compares the measured value with a threshold value. If the measured value is equal to or greater than the threshold value, the control unit 13 determines that the biological sound collecting device 1 is pressed against the biological surface H31 (hereinafter, referred to as a contact state). Additionally, if the measured value is smaller than the threshold value, the control unit 13 determines that the biological sound collecting device 1 is not pressed against the biological surface H31 (hereinafter, referred to as a non-contact state). Then, in accordance with state information including information representing the contact state and information representing the non-contact state, the control unit 13 outputs an instruction signal to the information notification unit 14. The state information includes sensor identification information that can identify each sensor configuring the contact state detecting unit 12.

Additionally, if the state information includes only information representing the contact state, that is, if the contact surface SM is properly pressed against the biological surface H31, the control unit 13 outputs an initiation signal that has the sound collecting unit 15 initiate collection of biological sound.

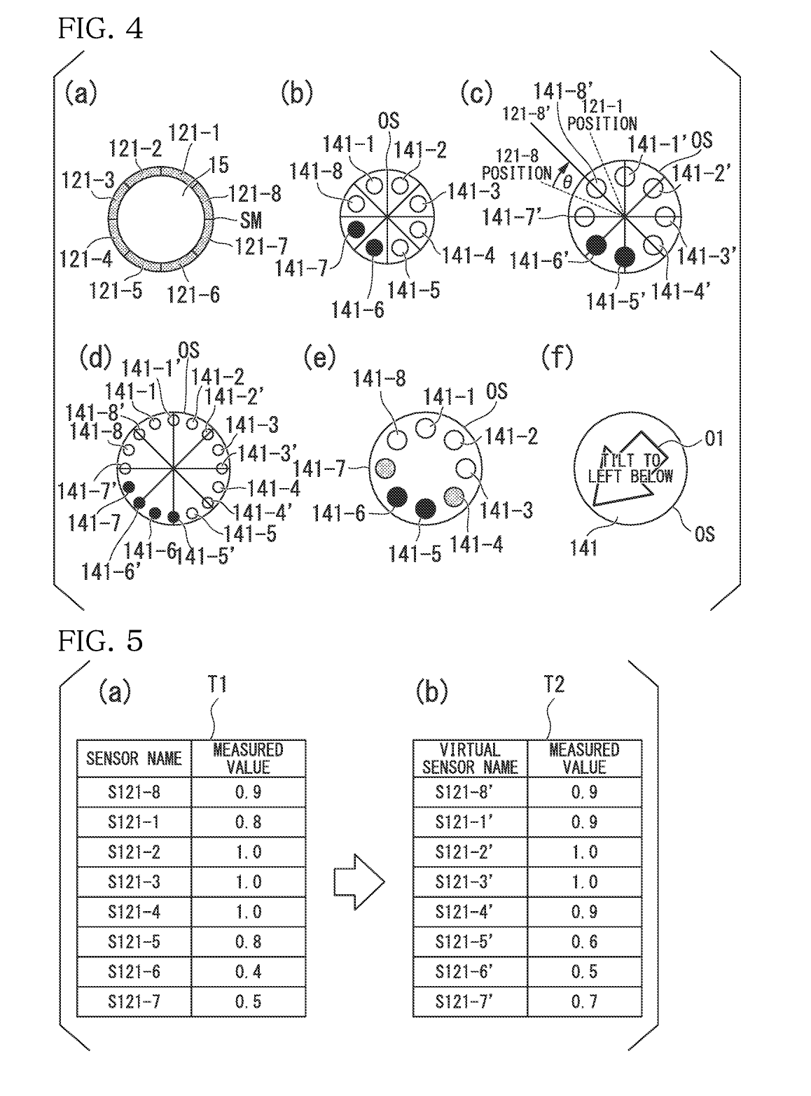

The presentation unit 141 includes a plurality of LEDs (light emitting elements: Light Emitting Diodes). Based on the instruction signal in accordance with the state information received from the control unit 13, the presentation unit 141 notifies the user of the pressing state of the biological sound collection device 1 against the biological surface H31. Specifically, based on the instruction signal in accordance with the state information, the presentation unit 141 turns on or off the LEDs corresponding to the respective sensors configuring the contact state detecting unit 12. Detailed description of the presentation unit 141 will be described later with reference to FIG. 4.

The sound collecting unit 15 has a diaphragm (not shown). By being held in contact with the biological surface H31 of the subject H3, the diaphragm vibrates in response to biological sound of the subject H3.

Based on the initiation signal received from the control unit 13, the microphone 151 collects biological sound by vibration of the diaphragm, and generates an analog signal of the collected biological sound. The microphone 151 outputs the generated analog signal to the A/D unit 152.

The A/D unit 152 converts the analog signal generated by the microphone 151 into a digital signal.

The A/D unit 152 outputs the converted digital signal to the data processing unit 16.

The data processing unit 16 quantizes the digital signal received from the A/D unit 152. The data processing unit 16 performs data processing, such as change of the frequency characteristics of a digital signal, enhancement or reduction of a particular frequency band of a digital signal. Additionally, in order to separate the biological sound and noise, the data processing unit 16 performs on the digital signal, data processing of separating the biological sound and noise, using a bandpass filter tailored to a frequency band of biological sound, such as 20 Hz to 600 Hz. The data processing unit 16 outputs to the D/A unit 21, the digital signal subjected to data processing.

The D/A unit 21 converts into an analog signal, the digital signal representing the biological sound received from the data processing unit 16. The D/A unit 21 outputs the converted analog signal to the speaker 22.

The speaker 22 notifies the user of the analog signal representing the biological sound received from the D/A unit 21.

FIG. 4 is a schematic diagram showing an example of a notification aspect of the state information in the presentation unit 141 of the biological sound collecting device 1 according to the first embodiment of the present invention.

FIG. 4(a) is similar to the arrangement example of the sensor shown in FIG. 2(c), and therefore description thereof is omitted here. Here, a case where the control unit 13 determines that the sixth sensor 121-6 and the seventh sensor 121-7 are in the non-contact state is described with reference to FIG. 4(b) to FIG. 4 (f).

FIG. 4(b) shows an example of a case where the arrangement positions of the respective sensors of the contact state detecting unit 12 on the contact surface SM of the biological sound collecting device 1 substantially match the arrangement positions of the respective LEDs on the information notification surface OS.

As shown in FIGS. 4(a) and 4(b), the first sensor 121-1 corresponds to the first LED 141-1, the second sensor 121-2 corresponds to the second LED 141-2, and the third sensor 121-3 corresponds to the third LED 141-3. Thus, each sensor configuring the contact state detecting unit 12 and each LED configuring the LED group are in correspondence with each other.

As described above, the control unit 13 determines that the sixth sensor 121-6 and the seventh sensor 121-7 are in the non-contact state, and turns on the sixth LED 141-6 and the seventh LED 141-7 corresponding to the sixth sensor 121-6 and the seventh sensor 121-7.

Thus, the user can confirm in real time, by the information notification unit 14, the pressing state of the contact surface SM of the biological sound collecting device 1 against the biological surface H31. If the pressing manner of the contact surface SM is not proper, the user can easily modify the pressing manner.

FIG. 4(c) shows an example of a case where the arrangement position of each sensor of the contact state detecting unit 12 on the contact surface SM of the biological sound collecting device 1 is deviated by angle θ from the arrangement position of each LED on the information notification surface OS.

For example, in a case, as shown in FIG. 4(c), where the LED corresponding to the eighth sensor 121-8 is placed at the position rotated by angle θ from the arrangement position of the eighth sensor 121-8 shown in FIG. 4(a), the control unit 13 may calculate an estimated value based on the measured values obtained by the respective sensors of the contact state detecting unit 12 and the following equation.

When θ>0,

(S121-8′)=(S121-8)×(1−t)+(S121-1)×t (1)

t=θ/(360 deg/number of divided regions)

Here, S121-8′ represents an estimated value of a virtual eighth sensor 121-8′. S121-8 represents a measured value of the eighth sensor 121-8. S121-1 represents a measured value of the first sensor 121-1. The number of divided regions represents the number of regions into which the information notification surface OS is divided, which is eight in the first embodiment.

When θ<0,

(S121-8′)=(S121-8)×(1−t)+(S121-7)×t (2)

t=−θ/(360 deg/number of divided regions)

Here, S121-8′ represents an estimate value of the virtual eighth sensor 121-8′. S121-8 represents a measured value of the eighth sensor 121-8. S121-7 represents a measured value of the seventh sensor 121-7. The number of divided regions represents the number of regions into which the information notification surface OS is divided, which is eight in the first embodiment.

In the example shown in FIG. 4(c), the number of divided regions is the eight division, and θ=22.5 deg. Based on the measured values obtained by the respective sensors of the contact state detecting unit 12 and the above equation, the control unit 13 calculates an estimated value as follows.

$$\begin{matrix}( S 121 \text{-} {8}^{′} ) = ( S 121 \text{-} 8 ) \times ( 1 - 22.5 / 45 ) + ( S 121 \text{-} 1 ) \times ( 22.5 / 45 ) \\ = ( ( S 121 \text{-} 8 ) + ( S 121 \text{-} 1 ) ) / 2\end{matrix}$$

Thus, when the arrangement position of each sensor of the contact state detecting unit 12 is deviated by angle θ from the arrangement position of each LED on the information notification surface OS, the control unit 13 calculates, regarding the LED as a reference, an estimated value based on the measured values obtained by the contact state detecting unit 12. Then, based on that estimated value, the control unit 13 turns on or off the LEDs of the presentation unit 141. A relationship between the measured value measured by the contact state detecting unit 12 and the estimated value (θ=22.5 deg) calculated by the control unit 13 is shown in FIG. 5.

FIG. 5 is a table showing an example of the relationship between the measured value measured by the contact state detecting unit 12 and the estimated value calculated by the control unit 13 according to the first embodiment of the present invention.

As shown in FIG. 5(a), a table T1 is a table in two-dimensional table format including rows and columns and having item columns representing a sensor name and a measured value.

In each row of the table T1, a sensor name and a measured value of the sensor corresponding to that sensor name are stored. Here, the measured value is a measured value obtained by normalizing a change in capacitance.

Additionally, as shown in FIG. 5(b), a table T2 is a table in two-dimensional table format including rows and columns and having item columns corresponding to a virtual sensor name and a measured value.

In each row of the table T2, virtual sensor names S121-8′, S121-1′, S121-2′, S121-3′, S121-4′, S121-5′, S121-6′, and S121-7′ corresponding respectively to the LEDs 141-1′ 141-2′, 141-3′, 141-4′, 141-5′, 141-6′, 141-7′, and 141-8′, and estimated values calculated by the control unit 13 corresponding to the virtual sensors are stored.

Referring back to FIG. 4, FIG. 4(d) shows a case where the presentation unit 141 includes LEDs, the number of which is greater than, for example, twice, the number of sensors configuring the contact state detecting unit 12.

Using the above equation (1) or (2), the control unit 13 calculates an estimated value from the measured values measured by the respective sensors of the contact state detecting unit 12. Based on the measured values and the estimated value calculated, the control unit 13 turns on or off the LEDs of the presentation unit 141.

Thus, the control unit 13, even if the presenting part 141 has the larger number of LEDs than the number of sensors of the contact state detecting unit 12, the user can confirm in real time, by the information notification unit 14, the pressing state of the contact surface SM of the biological sound collecting device 1 against the biological surface H31. If the pressing manner of the contact surface SM is not proper, the user can easily modify the pressing manner.

FIG. 4(e) shows an example of a case where the presentation unit 141 changes the tone of the lighting LEDs, thus notifying the pressing state.

The control unit 13 compares the measured value and a gradual threshold value. For each threshold value, the control unit 13 determines whether or not the measured value is within a range of the threshold value. Then, based on a result of the determination and its gradual threshold value, the control unit 13 may change the tone of the LED of the presentation unit 141 corresponding to the sensor having measured the measured value. In this case, it is sufficient for the presentation unit 141 to notify the state information by, for example, the three-level tone.

FIG. 4(f) shows an example of a case where the presentation unit 141 includes a display unit in place of the LEDs.

The presentation unit 141 includes a display unit, such as a liquid crystal display, an organic EL display, or a plasma display. The control unit 13 may, in accordance with the measured values of the respective sensors of the contact state detecting unit 12, have the presentation unit 141 display an object O1 representing an instruction to modify the pressing manner of the contact surface SM against the biological surface H31. In this case, the presentation unit 141 may have the display unit display, for example, the object O1 representing an arrow, in the direction corresponding to the position of the sensor in the non-contact state, and have the display unit display a character string “Please tilt to the lower left” corresponding to the object O1, thus notifying the user of the pressing manner.

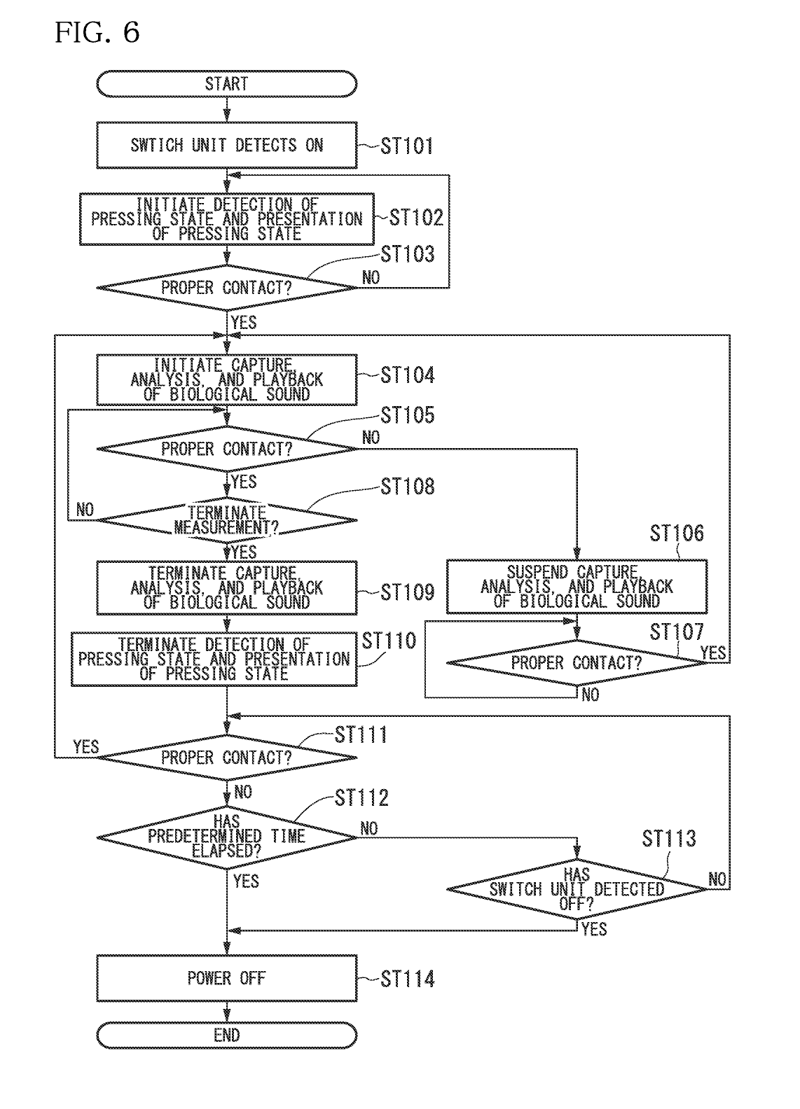

FIG. 6 is a flowchart showing an example of a pressing state presentation processing of the biological sound collecting device 1 according to the first embodiment of the present invention.

In step ST101, the switch unit 11 outputs to the control unit 13, an operation signal indicating “ON” by user operation.

In step ST102, the control unit 13 has the contact state detecting unit 12 and the presentation unit 141 initiate detection and presentation of a pressing state of the contact surface SM against the biological surface H31.

In step ST103, the control unit 13 determines whether or not the contact surface SM is properly in contact with the biological surface H31, that is, whether or not the state information includes only information indicating a contact state. If the state information includes only information indicating a contact state, the processing proceeds to step ST104. On the other hand, if the state information includes not only information indicating a contact state, the processing returns to step ST102.

In step ST104, the control unit 13 has the sound collecting unit 15 initiate capture, analysis and playback of the biological sound.

In step ST105, the control unit 13 determines whether or not the contact surface SM is properly in contact with the biological surface H31, that is, whether or not the state information includes only information indicating a contact state. If the state information includes only information indicating a contact state, the processing proceeds to step ST108. On the other hand, if the state information includes not only information indicating a contact state, the processing returns to step ST106.

In step ST106, the control unit 13 has the sound collecting unit 15 suspend the capture, analysis, and playback of the biological sound.

In step ST107, the control unit 13 determines whether or not the contact surface SM is properly in contact with the biological surface H31, that is, whether or not the state information includes only information indicating a contact state. If the state information includes only information indicating a contact state, the processing returns to step ST104. On the other hand, if the state information includes not only information indicating a contact state, step ST107 is repeated.

In step ST108, the control unit 13 determines whether or not the contact surface SM has been properly in contact with the biological surface H31 for a predetermined time, for example, 5 seconds, required for the capture, analysis, and playback of the biological sound. If the contact surface SM has been properly in contact with the biological surface H31, the measurement of the biological sound is terminated, and the processing proceeds to step ST109. On the other hand, if the contact surface SM has not been properly in contact with the biological surface H31, the processing returns to step ST105.

In step ST109, the control unit 13 has the sound collecting unit 15 terminate the capture, analysis, and playback of the biological sound.

In step ST110, the control unit 13 has the contact state detecting unit 12 and the presentation unit 141 terminate the detection and presentation of the pressing state of the contact surface SM against the biological surface H31.

In step ST111, the control unit 13 again determines whether or not the contact surface SM is properly in contact with the biological surface H31, that is, whether or not the state information includes only information indicating a contact state. If the state information includes only information indicating a contact state, the processing returns to step ST104, and capture, analysis, and playback of biological sound are initiated. On the other hand, if the state information includes not only information indicating a contact state, the processing proceeds to step ST112.

In step ST112, it is determined whether a predetermined time has elapsed since the measurement is completed. If the predetermined time has elapsed, the processing proceeds to step ST114. On the other hand, if the predetermined time has not yet elapsed, the processing proceeds to step ST113.

In step ST113, the control unit 13 determines whether an operation signal indicating “OFF” is inputted by user operation to the switch unit 11. If the operation signal indicating “OFF” is inputted, the processing proceeds to step ST114. On the other hand, if the operation signal indicating “OFF” is not inputted, the processing returns to step ST111.

In step ST114, the control unit 13 turns off the power, and terminates the pressing state presentation processing.

Thus, according to the first embodiment, the biological sound collecting device 1 includes the sound collecting unit 15, the contact state detecting unit 12, the control unit 13, and the information notification unit 14. The sound collecting unit 15 collects biological sound of the subject H3. The contact state detecting unit 12 detects contact with the biological surface H31 of the subject H3. Based on the contact of the subject H3 with the biological surface H31 detected by the contact state detecting unit 12, the control unit 13 determines whether or not to have the sound collecting unit 15 collect biological sound. If the control unit 13 determines not to have the sound collecting unit 15 collect biological sound, the information notification unit 14 notifies that the contact state detecting unit 12 is not in contact with the biological surface H31 of the subject H3.

As a result, the user can confirm in real time, by the information notification unit 14, the pressing state of the contact surface SM of the biological sound collecting device 1 against the biological surface H31. Additionally, if the pressing manner of the contact surface SM is not proper, it is possible to easily modify the pressing manner. For this reason, the user can accurately auscultate biological sound of the subject H3.

### Second Embodiment

Hereinafter, a second embodiment of the present invention will be described in detail with reference to the accompanying drawings.

The description has been given in the first embodiment with respect to the case where the information notification unit 14 of the biological sound collecting device 1 visually notifies the pressing state of the contact surface SM against the biological surface H31. Description will be given in the second embodiment with respect to a case where an information notification unit 14A of the biological sound collecting device 1A aurally notifies a pressing state of the contact surface SM against the biological surface H31.

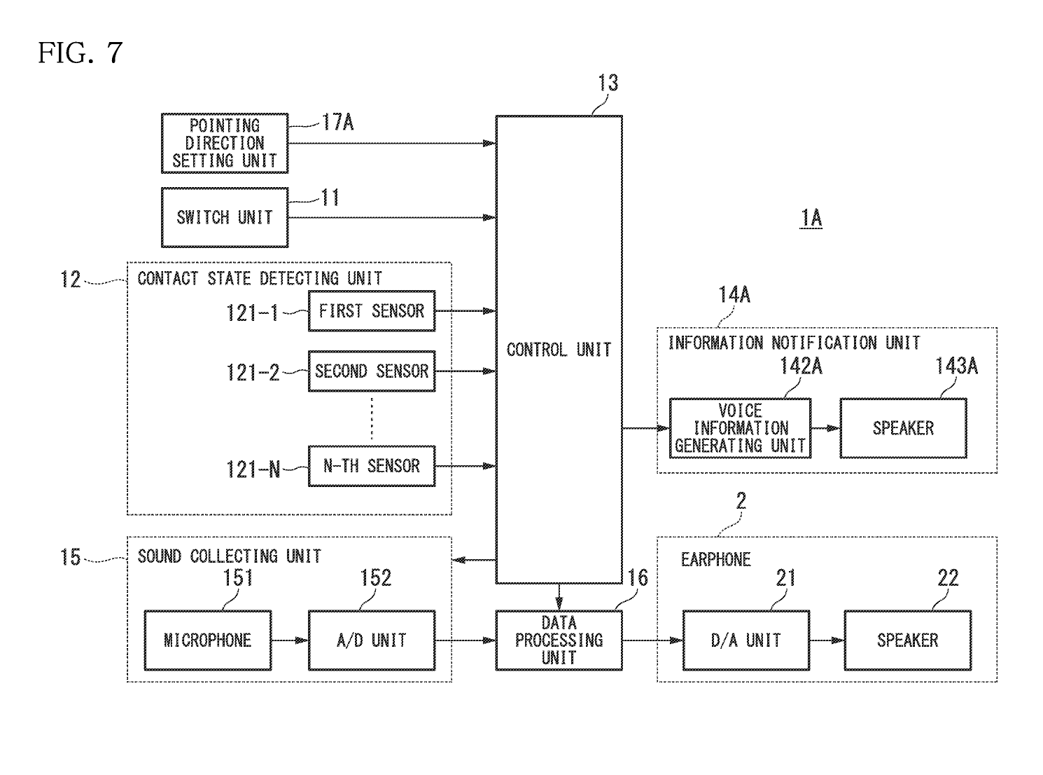

FIG. 7 is a schematic block diagram showing an example of a configuration of a biological sound collecting device 1A according to a second embodiment of the present invention.

Comparing the configuration of the biological sound collecting device 1 of the first embodiment shown in FIG. 3 with the configuration of the biological sound collecting device 1A of the second embodiment, a pointing direction setting unit 17A is added. Additionally, a configuration of an information notification unit 14A is different. Other configurations are similar to those of the biological sound collecting device 1, and therefore are appended with the same reference numerals, and description thereof is omitted here.

The pointing direction setting unit 17A has, for example, a ring shape. The centers of the information notification surface OS and the contact surface SM of the biological sound collecting device 1A match the center of the pointing direction setting unit 17A. The pointing direction setting unit 17A is provided inside a surface of a housing 10A of the biological sound collecting device 1A. The pointing direction setting unit 17A has, at a part of the ring, a reference pointing unit serving as a reference for a pointing direction. The pointing direction setting unit 17A is disposed with a structure that is rotatable by a user. The pointing direction setting section 17A outputs to the control unit 13, information representing a position of the reference pointing unit.

The information notification unit 14A includes a voice information generating unit 142A and a speaker 143A.

Based on the state information and the information representing the position of the reference pointing unit which is received from the control unit 13, the voice information generating unit 142A generates a voice signal to notify a position (direction) of a sensor in the non-contact state. The voice information generating unit 142A aurally notifies the user, by the generated audio signal via the speaker 143A, of the pressing state of the contact surface SM against the biological surface H31.

For example, in a case where a direction in which the reference pointing unit is positioned is regarded as a front, and a sensor at the front configuring the contact state detecting unit 12 is a non-contact state, the voice information generating unit 142A generates a voice signal indicating “Please tilt forward.” Additionally, in a case where a right side direction with respect to the reference pointing unit is regard as the right of the biological sound collecting device 1A, and a sensor at the right configuring the contact state detecting unit 12 is in the non-contact state, the voice information generating unit 142A generates a voice signal indicating “Please tilt to the right.” Further, in a case where a direction opposite to the direction of the reference pointing unit is regarded as the front, and a sensor at the front configuring the contact state detecting unit 12 is in the non-contact state, the voice information generating unit 142A generates a voice signal indicating “Please tilt to the front.” Moreover, in a case where a left side direction with respect to the reference pointing unit is regarded as the left of the biological sound collecting device 1A, a sensor at the left configuring the contact state detecting unit 12 is in the non-contact state, the voice information generating unit 142A generates a voice signal indicating “Please tilt to the left.”

Here, although an example of the case where the pointing direction setting unit 17A has the ring shape has been shown, and the means of setting the pointing direction has been described, the present embodiment is not limited thereto. For example, a configuration may be such that the information notification unit 14A includes the presentation unit 141 as in the first embodiment, and the pointing direction setting unit 17A has the presentation unit 141 display a screen for setting a pointing direction, and receive a setting of the pointing direction from the setting screen. Additionally, a configuration may be such that the pointing direction setting unit 17A has the LEDs of the presentation unit 141 cooperate with the respective sensors 121 of the contact state detecting unit 12, and receive a setting of the pointing direction in accordance with the sensor position of the sensor 121 having detected contact of the contact state detecting unit 12.

Here, a configuration may be such that the presentation unit 141 of the information notification unit 14 according to the first embodiment, and the voice information generating unit 142A and the speaker 143A of the information notification unit 14A according to the second embodiment are combined, so as to visually and aurally notify the user of the pressing state of the contact surface SM against the biological surface H31.

Thus, according to the second embodiment, the biological sound collecting device 1A includes the sound collecting unit 15, the contact state detecting unit 12, the control unit 13, and the information notification unit 14A. The sound collecting unit 15 collects biological sound of the subject H3. The contact state detecting unit 12 detects contact with the biological surface H31 of the subject H3. Based on the contact with the biological surface H31 of the subject H3 detected by the contact state detecting unit 12, the control unit 13 determines whether or not to have the sound collecting unit 15 collect biological sounds. If the control unit 13 determines not to have the sound collecting unit 15 collect the biological sound, the information notification unit 14A notifies that the contact state detecting unit 12 is not in contact with the biological surface H31 of the subject H3.

As a result, the user can confirm in real time, by the information notification unit 14A, the pressing state of the contact surface SM of the biological sound collecting device 1A against the biological surface H31. Thus, if the pressing manner of the contact surface SM of the biological sound collecting device 1A against the biological surface H31 is not proper, the pressing manner can be modified easily. For this reason, it is possible to accurately auscultate biological sound of the subject H3. Additionally, even if it is difficult to visually confirm the pressing state, it is possible to aurally notify the pressing state.

### Third Embodiment

Hereinafter, a third embodiment of the present invention will be described in detail with reference to the accompanying drawings.

In the third embodiment, description will be given with respect to a case where an information notification unit 14B of a biological sound collecting device 1B tactually notifies a pressing state of the contact surface SM against the biological surface H31.

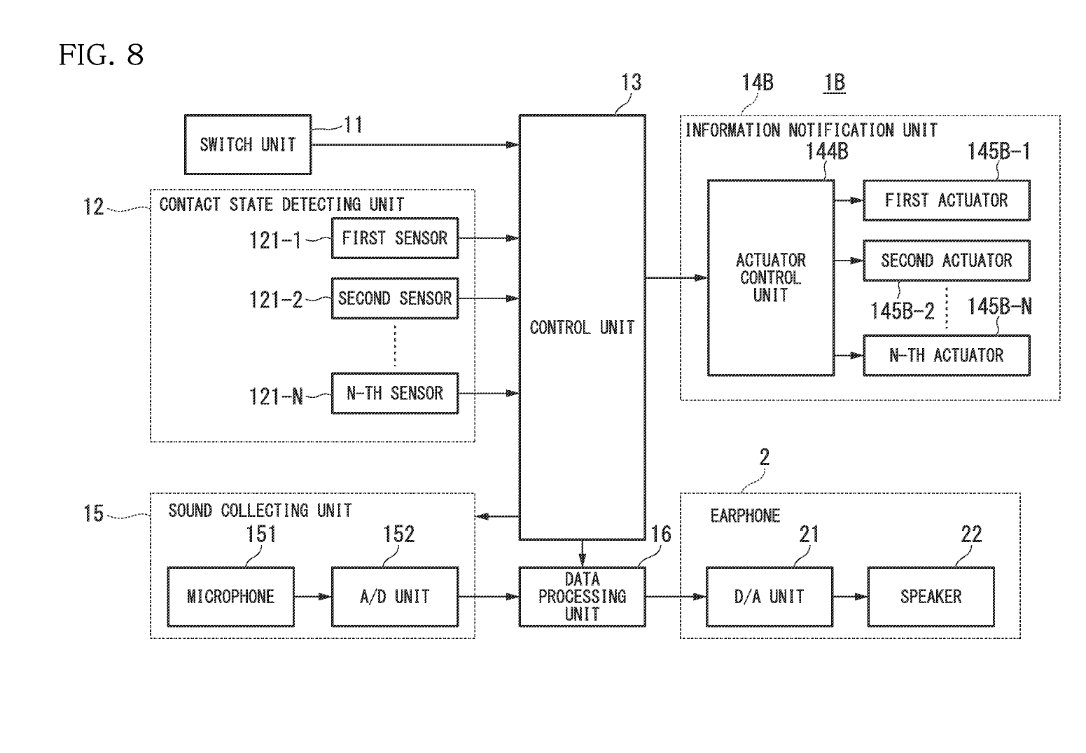

FIG. 8 is a schematic block diagram showing an example of a configuration of the biological sound collecting device 1B according to the third embodiment of the present invention.

Comparing the configuration of the biological sound collecting device 1B according to the third embodiment with the configuration of the biological sound collecting device 1 according to the first embodiment, a configuration of an information notification unit 14B is different. Other configurations are similar to those of the first embodiment, and therefore appended with the same reference numerals, and description thereof is omitted here.

The information notification unit 14B includes an actuator control unit 144B, a first actuator 145B-1, a second actuator 145B-2, . . . , and an N-th actuator 145B-N.

The actuator control unit 144B controls the first actuator 145B-1, the second actuator 145B-2, . . . , and the N-th actuator 145B-N(N is an integer). Specifically, based on state information received from the control unit 13, the actuator control unit 144B raises an actuator corresponding to a non-contact sensor of the contact state detecting unit 12, and tactually notifies a user of the pressing state of the contact surface SM against the biological surface H31.

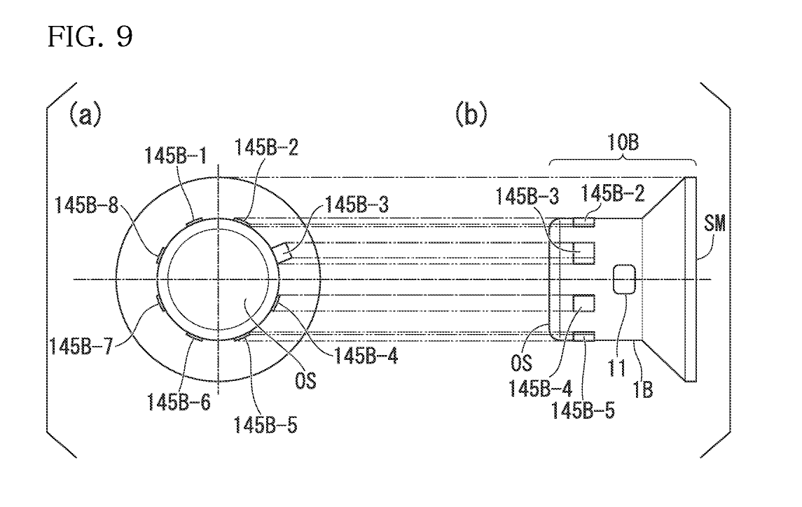

FIG. 9(a) is an example of a top view when the biological sound collecting device 1B according to the third embodiment of the present invention is viewed from the information notification surface OS. FIG. 9(b) is an example of a side view of the biological sound collecting device 1B.

In FIG. 9(a), a plurality of actuators are circularly arranged on a periphery of the information notification surface OS of a housing 10B of the biological sound collecting device 1B. For example, eight actuators which are the first actuator 145B-1, the second actuator 145B-2, the third actuator 145B-3, the fourth actuator 145B-4, the fifth actuator 145B-5, the sixth actuator 145B-6, the seventh actuator 145B-7, and the eighth actuator 145B-8, are arranged around the information notification surface OS.

The example of FIG. 9(a) is an example where the third sensor 121-3 of the contact state detecting unit 12 is in the non-contact state, and the third the actuator 145B-3 corresponding to the third sensor 121-3 is raised.

In FIG. 9(b), the switch unit 11 is disposed in the housing 10B between the contact surface SM and the information notification surface OS of the biological sound collecting device 1B. The plurality of actuators are arranged between the switch unit 11 and the information notification surface OS. For example, the second actuator 145B-2, the third actuator 145B-3, the fourth actuator 145B-4, and the fifth actuator 145B-5 are arranged on the side surface of the housing 10B of the biological sound collecting device 1B.

Here, the number of actuators is preferably the same as the number of sensors configuring the contact state detecting unit 12. However, even in a case where the number of sensors constituting the contact state detecting unit 12 differs from the number of actuators, it is possible to notify, from the measured values and the estimated value, the pressing state of the contact surface SM against the biological surface H31, as shown in FIG. 4(d).

Additionally, a configuration may be such that the presentation unit 141 of the information notification unit 14 according to the first embodiment, and the actuator control unit 144B and the plurality of actuators 145B of the information notification unit 14B according to the third embodiment are combined so as to visually and tactually notify the user of the pressing state of the contact surface SM against the biological surface H31.

Here, a configuration may be such that the voice information generating unit 142A and the speaker 143A of the information notification unit 14A according to the second embodiment, and the actuator control unit 144B and the plurality of actuators 145B of the information notification unit 14B according to the third embodiment are combined so as to aurally and tactually user notify the user of the pressing state of the contact surface SM against the biological surface H31.

Additionally, a configuration may be such that the presentation unit 141 of the information notification unit 14 according to the first embodiment, the voice information generating unit 142A and the speaker 143A of the information notification unit 14A according to the second embodiment, and the actuator control unit 144B and the plurality of actuators 145B of the information notification unit 14B according to the third embodiment are combined so as to visually, aurally, and tactually notify the user of the pressing state of the contact surface SM against the biological surface H31.

Thus, according to the third embodiment, the biological sound collecting device 1B includes the sound collecting unit 15, the contact state detecting unit 12, the control unit 13, and the information notification unit 14B. The sound collecting unit 15 collects biological sound of the subject H3. The contact state detecting unit 12 detects contact with the biological surface H31 of the subject H3. Based on the contact with the biological surface H31 of the subject H3 detected by the contact state detecting unit 12, the control unit 13 determines whether or not to have the sound collecting unit 15 collect biological sound. If the control unit 13 determines not to have the sound collecting unit 15 collect biological sound, the information notification unit 14B notifies that the contact state detecting unit 12 is not in contact with the biological surface H31 of the subject H3.

As a result, the user can confirm in real time, by the information notification unit 14B, the pressing state of the contact surface SM of the biological sound collecting device 1B against the biological surface H31. Additionally, if the pressing manner of the contact surface SM is not proper, it is possible to modify the pressing manner easily. For this reason, it is possible to accurately auscultate biological sound of the subject H3. Further, even if it is difficult to visually and aurally confirm the pressing state of the contact surface SM, it is possible to tactually confirm the pressing state.

Here, although it has been described in the first to third embodiments that the sound collecting unit 15 and the contact state detecting unit 12 are disposed on the contact surface SM, those embodiments are not limited thereto. For example, as long as the contact state detecting unit 12 can detect a pressing state of the sound collecting unit 15 against the biological surface H31, the sound collecting unit 15 and the contact state detecting unit 12 may not be disposed on the same plane.

Here, the description has been given in the first to third embodiments with respect to the case where the contact state detecting unit 12 is disposed on the periphery of the sound collecting unit 15, those embodiments are not limited thereto. For example, the contact state detecting unit 12 may not be disposed on the periphery of the sound collecting unit 15 as long as the position of the contact state detecting unit 12 is a position not affecting the diaphragm included in the sound collecting unit 15 and enabling detection of the pressing state of the sound collecting unit 15 against the biological surface H31.

Here, in the first to third embodiments described above, a configuration may be such that reference pointing units which project outward from the housings 10, 10A, and 10B of the biological sound collecting devices 1, 1A, and 1B are included, and a pointing direction may be notified based on those reference pointing units.

Here, in the first to third embodiments described above, the contact state detecting unit 12 may be a sensor of piezo-resistive-type, a sensor of surface acoustic wave type, or an infrared sensor. In other words, it is sufficient that the contact state detecting unit 12 is a sensor capable of detecting pressing or contact. Additionally, the threshold value used for the control unit 13 to perform the determination described above may be common to all the sensors configuring the contact state detecting unit 12. Alternatively, the different sensors configuring the contact state detecting unit 12 may use different threshold values.

Additionally, although it has been described that the periphery of the sound collecting unit 15 is divided into the eight regions, and the sensors are placed on the respective regions, the embodiments are not limited thereto. For example, one sensor may be disposed on the periphery without dividing the periphery region of the sound collecting unit 15. Further, the periphery region of the sound collecting unit 15 may be divided into two or more regions so that sensors are disposed on the respective regions. Moreover, the D/A unit 21 has been described as a component of the earphone 2, but, for example, the biological sound collecting device 1 may include the D/A unit 21.

Here, in the first to third embodiments described above, a configuration may be such that the biological sound collecting device 1, 1A, and 1B include memory to store a digital signal obtained by the data processing unit 16 performing data processing on the biological sound collected by the sound collecting unit 15.

Here, the biological sound collecting devices 1, 1A, and 1B according to the first to third embodiments described above may be realized in part or whole by a computer. In that case, the biological sound collecting devices 1, 1A, and 1B may be realized by recording a program for realizing the control functions thereof on a computer-readable recording medium, and having a computer system read and execute the program recorded on the recording medium. Here, the “computer system” herein means a computer system built in the biological sound collecting devices 1, 1A, and 1B, and includes an OS and hardware, such as peripheral devices.

Additionally, the “computer-readable recording medium” means a portable medium such as a flexible disk, a magneto-optical disk, an ROM, and a CD-ROM, or a storage device such as a hard disk built in the computer system. Further, the “computer-readable recording medium” may include a medium that dynamically stores a program in a short time, like a communication line when the program is transmitted through a network such as the Internet, or through a communication line such as a telephone line. Moreover, the “computer-readable recording medium” may include a medium that stores the program for a given period, like a volatile memory inside the computer system serving as a server or client in the above case.

Additionally, the above program may be a program for realizing part of the above-described functions. Further, the above program may be one that can realize the above-described functions in combination with a program already recorded on the computer system.

The biological sound collecting device 1, 1A, and 1B according to the first to third embodiments described above may be realized in part or whole as an integrated circuit, such as an LSI (Large Scale Integration). Each functional block of the biological sound collecting devices 1, 1A, and 1B may be individually made into a processor. Alternatively, part or whole of the functional blocks of the biological sound collecting devices 1, 1A, and 1B may be integrated into a processor. Further, the circuit integrating method is not limited to LSI, and circuit integration may be realized by a dedicated circuit or a general-purpose processor. Moreover, if integrated circuit technology that replaces the LSI emerges along with the advancement of the semiconductor technology, an integrated circuit realized by that technology may be used.

Although the first to third embodiments of the present invention have been described in detail with reference to the drawings, the specific configurations are not limited to those described above, and various design changes and the like can be made without departing from the scope of the present invention.

(1) A biological sound collecting device includes: a sound collecting unit configured to collect biological sound of a subject; a contact state detecting unit configured to detect a contact state of the sound collecting unit against a biological surface of the subject; a control unit configured to determine, based on the contact state detected by the contact state detecting unit, whether or not to have the sound collecting unit collect the biological sound; and an information notification unit configured to, in a case that the control unit determines not to have the sound collecting unit collect the biological sound, notify that the contact state detecting unit is not in contact with the biological surface of the subject.

(2) Regarding the biological sound collecting device according to the above (1), the contact state detecting unit includes a plurality of sensors, the plurality of sensors are arranged at different positions on a periphery of the sound collecting unit, and the information notification unit is configured to notify a position of a sensor, of the plurality of sensors, which has not detected contact with the biological surface of the subject.

(3) Regarding the biological sound collecting device according to the above (2), the information notification unit includes a plurality of light emitting elements, and the information notification unit is configured to turn on the light emitting element corresponding to the position of the sensor, of the plurality of sensors, which has not detected contact with the biological surface of the subject.

(4) Regarding the biological sound collecting device according to the above (2), the information notification unit includes a display unit, and the information notification unit is configured to have the display unit display the position of the sensor, of the plurality of sensors, which has not detected contact with the biological surface of the subject.

(5) The biological sound collecting device according to the above (2) further includes: a pointing direction setting unit configured to determine a reference pointing direction at the time of notifying the position of the sensor which has not detected contact with the biological surface of the subject. The information notification unit is configured to, in accordance with the reference pointing direction determined by the pointing direction setting unit, aurally notify the position of the sensor, of the plurality of sensors, which has not detected contact with the biological surface of the subject.

(6) Regarding the biological sound collecting device according to the above (2), the information notification unit comprises a plurality of actuators provided on a housing of the biological sound collecting device, and the information notification unit is configured to, in order to perform notification, raise the actuator corresponding to the position of the sensor, of the plurality of sensors, which has not detected contact with the biological surface of the subject.

(7) A biological sound collecting method includes: collecting, by a sound collecting unit, biological sound of a subject; detecting, by a contact state detecting unit, a contact state of the sound collecting unit against a biological surface of the subject; determining, by a control unit, based on the contact state detected by the contact state detecting unit, whether or not to have the sound collecting unit collect the biological sound; and in a case that the control unit determines not to have the sound collecting unit collect the biological sound, notifying, by an information notification unit, that the contact state detecting unit is not in contact with the biological surface of the subject.

### INDUSTRIAL APPLICABILITY

One aspect of the present invention is applicable to biological sound collecting devices and biological sound collecting methods, which require accurate auscultation of biological sound of a subject.

### DESCRIPTION OF REFERENCE NUMERALS

1, 1A, 1B: biological sound collecting device
10, 10A, 10B: housing
11: switch unit
12: contact state detecting unit
121-1: first sensor
121-2: second sensor
121-3: third sensor
121-4: fourth sensor
121-5: fifth sensor
121-6: sixth sensor
121-7: seventh sensor
121-8: eighth sensor
121-N: N-th sensor
13: control unit
14, 14A, 14B: information notification unit
141: presentation unit
142A: voice information generating unit
143A: speaker
144B: actuator control unit
145B-1: first actuator
145B-2: second actuator
145B-3: third actuator
145B-4: fourth actuator
145B-5: fifth actuator
145B-N: N-th actuator
15: sound collecting unit
151: microphone
152: A/D unit
16: data processing unit
17A: pointing direction setting unit
2: earphone
21: D/A unit
22: speaker

1. A biological sound collecting device comprising:
a sound collecting unit configured to collect biological sound of a subject;
a contact state detecting unit configured to detect a contact state of the sound collecting unit against a biological surface of the subject;
a control unit configured to determine, based on the contact state detected by the contact state detecting unit, whether or not to have the sound collecting unit collect the biological sound; and
an information notification unit configured to, in a case that the control unit determines not to have the sound collecting unit collect the biological sound, notify that the contact state detecting unit is not in contact with the biological surface of the subject, wherein
the contact state detecting unit comprises a plurality of sensors,
the plurality of sensors are arranged at different positions on a periphery of the sound collecting unit, and
the information notification unit is configured to notify a position of a sensor, of the plurality of sensors, which has not detected contact with the biological surface of the subject.

2. (canceled)

3. The biological sound collecting device according to claim 1, wherein
the information notification unit comprises a plurality of light emitting elements, and
the information notification unit is configured to turn on the light emitting element corresponding to the position of the sensor, of the plurality of sensors, which has not detected contact with the biological surface of the subject.

4. The biological sound collecting device according to claim 1, wherein
the information notification unit comprises a display unit, and
the information notification unit is configured to have the display unit display the position of the sensor, of the plurality of sensors, which has not detected contact with the biological surface of the subject.

5. The biological sound collecting device according to claim 1, further comprising:
a pointing direction setting unit configured to determine a reference pointing direction at the time of notifying the position of the sensor which has not detected contact with the biological surface of the subject,
wherein the information notification unit is configured to, in accordance with the reference pointing direction determined by the pointing direction setting unit, aurally notify the position of the sensor, of the plurality of sensors, which has not detected contact with the biological surface of the subject.

6. The biological sound collecting device according to claim 1, wherein
the information notification unit comprises a plurality of actuators provided on a housing of the biological sound collecting device, and
the information notification unit is configured to, in order to perform notification, raise the actuator corresponding to the position of the sensor, of the plurality of sensors, which has not detected contact with the biological surface of the subject.

7. A biological sound collecting method comprising:
detecting, by a contact state detecting unit, a contact state of a sound collecting unit against a biological surface of the subject;
determining, by a control unit, based on the contact state detected by the contact state detecting unit, whether or not to have the sound collecting unit collect the biological sound; and
in a case that the control unit determines not to have the sound collecting unit collect the biological sound, notifying, by an information notification unit, a position of a sensor, of a plurality of sensors, which has not detected contact with the biological surface of the subject, thus notifying that the contact state detecting unit is not in contact with the biological surface of the subject,
wherein the plurality of sensors are included in the contact state detecting unit and arranged at different positions on a periphery of the sound collecting unit.
